(19) United States
(12) Patent Application Publication
(10) Pub. No.: US 2015/0166438 A1
(43) Pub. Date: Jun. 18, 2015

(54) PROCESSES AND APPARATUSES FOR ISOMERIZING HYDROCARBONS

(71) Applicant: UOP LLC, Des Plaines, IL (US)

(72) Inventor: Bryan K. Glover, Algonquin, IL (US)

(73) Assignee: UOP LLC, Des Plaines, IL (US)

(21) Appl. No.: 14/104,829

(22) Filed: Dec. 12, 2013

(51) Int. Cl. C07C 5/27

(52) U.S. Cl. CPC C07C 5/2732

(57) ABSTRACT

Processes and apparatuses for isomerizing hydrocarbons are provided. In an embodiment, a process for isomerizing hydrocarbons includes providing a first hydrocarbon feed that includes hydrocarbons having from 5 to 7 carbon atoms. The first hydrocarbon feed is fractionated to produce a first separated stream that includes hydrocarbons having from 5 to 6 carbon atoms and a second separated stream that includes hydrocarbons having 7 carbon atoms. The first separated stream is isomerized in the presence of a first isomerization catalyst and hydrogen under first isomerization conditions to produce a first isomerized stream. The second separated stream is isomerized in the presence of a second isomerization catalyst and hydrogen under second isomerization conditions that are different from the first isomerization conditions to produce a second isomerized stream. The first isomerization catalyst is the same type of isomerization catalyst as the second isomerization catalyst.

### TECHNICAL FIELD

The technical field generally relates to processes and apparatuses for isomerizing hydrocarbons. More particularly, the technical field relates to processes and apparatuses for separately isomerizing a stream containing C5 and C6 hydrocarbons, and a stream containing C7 hydrocarbons.

### BACKGROUND

Hydrocarbon streams are refined through various unit operations to produce various types of fuel, industrial raw materials that are employed in production of other compounds or products, and petroleum-based products. Production of gasoline is a particularly important industrial process involving refining of hydrocarbons through various unit operations, including isomerization and catalytic reforming. Reforming of hydrocarbons is useful to convert paraffins to aromatic compounds in the presence of noble metal catalysts. Aromatic compounds are associated with high octane values and, thus, are desirable components in gasoline. Isomerization is effective to convert linear hydrocarbons into branched hydrocarbons, which have a higher octane value than linear compounds but a lower octane value than aromatic compounds. Isomerized streams (or isomerate) is substantially free of aromatic compounds, whereas reformate streams (or reformate) generally include high quantities of aromatic compounds (e.g., at least 50 wt %).

During refining, a hydrocarbon stream that includes a range of hydrocarbons is generally separated into various streams based on the number of carbon atoms of compounds within the streams. Hydrocarbons having 7 or more carbon atoms are generally subject to reforming because reforming generally results in higher octane values than isomerization of these hydrocarbons. Hydrocarbons having 5 or 6 carbon atoms are generally subject to isomerization.

Modern specifications for gasoline typically place limits on aromatic content. The limits on aromatic content restricts the amount of reformate that can be blended into the gasoline. Since refineries generally produce significantly more hydrocarbons having 7 or more carbon atoms, there is typically too much reformate produced relative to isomerate for cases where aromatics are highly restricted in gasoline. Hydrocarbons having 7 carbon atoms cannot be effectively isomerized with hydrocarbons having 5 or 6 carbon atoms, with hydrocarbons having 7 carbon atoms subject to cracking under conditions necessary to effectively isomerize hydrocarbons having 5 or 6 carbon atoms. It has been suggested to isomerize a first separated stream having 5 or 6 carbon atoms and second separated stream having 7 carbon atoms in the presence of different isomerization catalysts, such as chlorided alumina for isomerizing the first separated stream having 5 or 6 carbon atoms and zirconia-containing catalyst to isomerize the second separated stream having 7 carbon atoms. However, chlorided alumina and zirconia-containing catalysts require additional and mutually exclusive unit operations that add significant cost and complexity. For example, chlorided alumina is sensitive to oxygen-containing compounds and requires a drying unit to remove water and other oxygen-containing compounds from the stream that is subject to isomerization, although the chlorided alumina can operate effectively without the need for hydrogen recycle. Zirconia-containing catalysts are not as sensitive as chlorided alumina to oxygen-containing compounds but generally incorporate a hydrogen recycle.

Accordingly, it is desirable to provide apparatuses and processes for isomerizing hydrocarbons that enable hydrocarbons having 5 or 6 carbon atoms and hydrocarbons having 7 carbon atoms to be separately and effectively isomerized while minimizing additional unit operations that are associated with various isomerization catalysts. Furthermore, other desirable features and characteristics of the present invention will become apparent from the subsequent detailed description of the invention and the appended claims, taken in conjunction with the accompanying drawings and this background of the invention.

### BRIEF SUMMARY

Processes and apparatuses for isomerizing hydrocarbons are provided. In an embodiment, a process for isomerizing hydrocarbons includes providing a first hydrocarbon feed that includes hydrocarbons having from 5 to 7 carbon atoms. The first hydrocarbon feed is fractionated to produce a first separated stream that includes hydrocarbons having from 5 to 6 carbon atoms and a second separated stream that includes hydrocarbons having 7 carbon atoms. The first separated stream is isomerized in the presence of a first isomerization catalyst and hydrogen under first isomerization conditions to produce a first isomerized stream. The second separated stream is isomerized in the presence of a second isomerization catalyst and hydrogen under second isomerization conditions that are different from the first isomerization conditions to produce a second isomerized stream. The first isomerization catalyst is the same type of isomerization catalyst as the second isomerization catalyst. The first isomerized stream and the second isomerized stream are stabilized together to produce a light stream that includes hydrocarbons having less than or equal to 4 carbon atoms and a stabilized stream that includes branched hydrocarbons.

In another embodiment, a process for isomerizing hydrocarbons includes fractionating a first hydrocarbon feed to produce a first separated stream that includes hydrocarbons having from 5 to 6 carbon atoms and a second separated stream that includes hydrocarbons having 7 carbon atoms. The first separated stream is isomerized in the presence of a first isomerization catalyst and hydrogen under first isomerization conditions to produce a first isomerized stream. The second separated stream is isomerized in the presence of a second isomerization catalyst and hydrogen under second isomerization conditions that are different from the first isomerization conditions to produce a second isomerized stream. The first isomerization catalyst is the same type of isomerization catalyst as the second isomerization catalyst. The first isomerized stream and the second isomerized stream are stabilized together to produce a light stream that includes hydrocarbons having less than or equal to 4 carbon atoms and a stabilized stream that includes branched hydrocarbons. The stabilized stream is fractionated into a first product stream that includes branched hydrocarbons having less than or equal to 6 carbon atoms and linear hydrocarbons having less than or equal to 5 carbon atoms, a normal hexane-enriched stream, and a second heavy fractionation stream that includes hydrocarbons having at least 7 carbon atoms. The normal hexane-enriched stream is combined with the first separated stream. The second heavy fractionation stream is fractionated into a second product stream that includes branched hydrocarbons having less than or equal to 7 carbon atoms, a normal heptane-enriched stream, and a third heavy fractionation stream that includes cyclic hydrocarbons having at least 7 carbon atoms. The normal heptane-enriched stream is combined with the second separated stream.

In another embodiment, an apparatus for isomerizing hydrocarbons includes a first fractionation unit that is adapted to fractionate a first hydrocarbon feed including hydrocarbons having from 5 to 7 carbon atoms to produce a first separated stream that includes hydrocarbons having from 5 to 6 carbon atoms and a second separated stream that includes hydrocarbons having 7 carbon atoms. A first isomerization unit is in fluid communication with the first fractionation unit and is adapted to receive and isomerize the first separated stream in the presence of a first isomerization catalyst and hydrogen under first isomerization conditions to produce a first isomerized stream. A second isomerization unit is in fluid communication with the first fractionation unit and is adapted to receive and isomerize the second separated stream in the presence of a second isomerization catalyst and hydrogen under second isomerization conditions different from the first isomerization conditions to produce a second isomerized stream. The first isomerization catalyst is the same type of isomerization catalyst as the second isomerization catalyst. A stabilizer is adapted to stabilize the first isomerized stream and the second isomerized stream together to produce a light stream that includes hydrocarbons having less than or equal to 4 carbon atoms and a stabilized stream that includes branched hydrocarbons.

### BRIEF DESCRIPTION OF THE DRAWINGS

The various embodiments will hereinafter be described in conjunction with the following drawing figures, wherein like numerals denote like elements, and wherein:

FIG. 1 is a schematic diagram of a process and an apparatus for isomerizing hydrocarbons in accordance with an exemplary embodiment;

FIG. 2 is a schematic diagram of a process and an apparatus for isomerizing hydrocarbons in accordance with another exemplary embodiment; and

FIG. 3 is a schematic diagram of a process and an apparatus for isomerizing hydrocarbons in accordance with yet another exemplary embodiment.

### DETAILED DESCRIPTION

The following detailed description is merely exemplary in nature and is not intended to limit the various embodiments or the application and uses thereof. Furthermore, there is no intention to be bound by any theory presented in the preceding background or the following detailed description.

Processes and apparatuses for isomerizing hydrocarbons are provided herein. The processes and apparatuses enable a first separated stream that includes hydrocarbons having 5 or 6 carbon atoms and a second separated stream that includes hydrocarbons having 7 carbon atoms to be separately and effectively isomerized while minimizing additional unit operations that are associated with various isomerization catalysts. In particular, a first isomerization catalyst is employed for isomerization of the first separated stream to produce a first isomerized stream and a second isomerization catalyst is employed for isomerization of the second separated stream to produce a second isomerized stream. The first isomerization catalyst is the same type of isomerization catalyst as the second isomerization catalyst, but the first separated stream and the second separated stream are subject to different isomerization conditions particular to the hydrocarbon species contained in the respective streams. By “same type”, it is meant that the first isomerization catalyst and the second isomerization catalyst are in the same class or family and benefit from the same supporting unit operations. For example, the “same type” of isomerization catalysts may be chlorided alumina in embodiments, or zirconia-containing catalyst in other embodiments. The first separated stream may be subject to slower space velocity and/or higher temperatures (so-called “higher severity” conditions) than the second separated stream, thereby avoiding cracking of the hydrocarbons having 7 carbon atoms while still effectively isomerizing the hydrocarbons having 5 or 6 carbon atoms. The first isomerized stream and the second isomerized stream are stabilized together, although intervening unit operations are possible such as a liquid/vapor separation to first separate hydrogen. Stabilization produces a light stream that includes hydrocarbons having less than or equal to 4 carbon atoms and a stabilized stream that includes branched hydrocarbons. By stabilizing the first isomerized stream and the second isomerized stream together, duplicitous unit operations for stabilizing the respective isomerized streams may be avoided. Further, process requirements differ for different classes of catalysts, and by using the same type of isomerization catalyst for the first isomerization catalyst and the second isomerization catalyst, process efficiencies may be maximized by employing common unit operations where possible. For example, because chlorided alumina catalysts generally require drying of the stream to be isomerized, in embodiments, a common dryer may be employed prior to isomerization of the respective separated streams, and other common unit operations may also be employed as described in further detail below. As another example, while processes and apparatuses that employ zirconia-containing isomerization catalysts generally do not incorporate drying of the stream to be isomerized, such processes and apparatuses generally incorporate hydrogen recycle from the resulting isomerized streams and a common recycled hydrogen stream may be employed to recycle hydrogen to both the first separated stream and the second separated stream prior to isomerization. Additional unit operations may be consolidated as described in further detail below, thereby maximizing processing efficiency and minimizing costs while enabling effective isomerization of C5, C6 and C7 hydrocarbons.

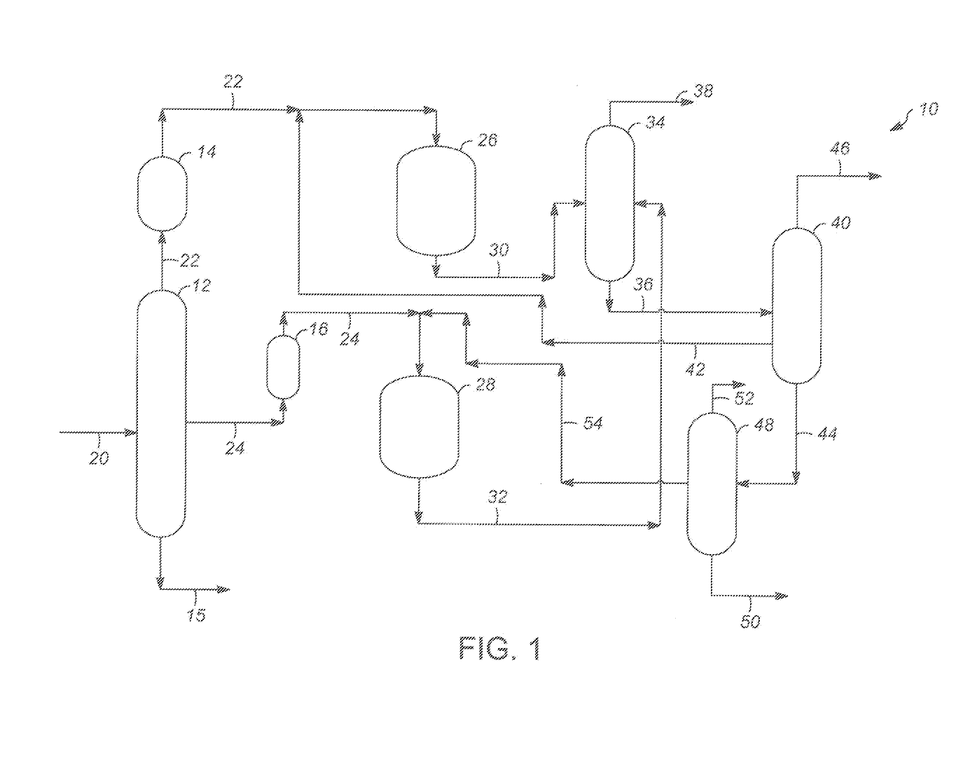

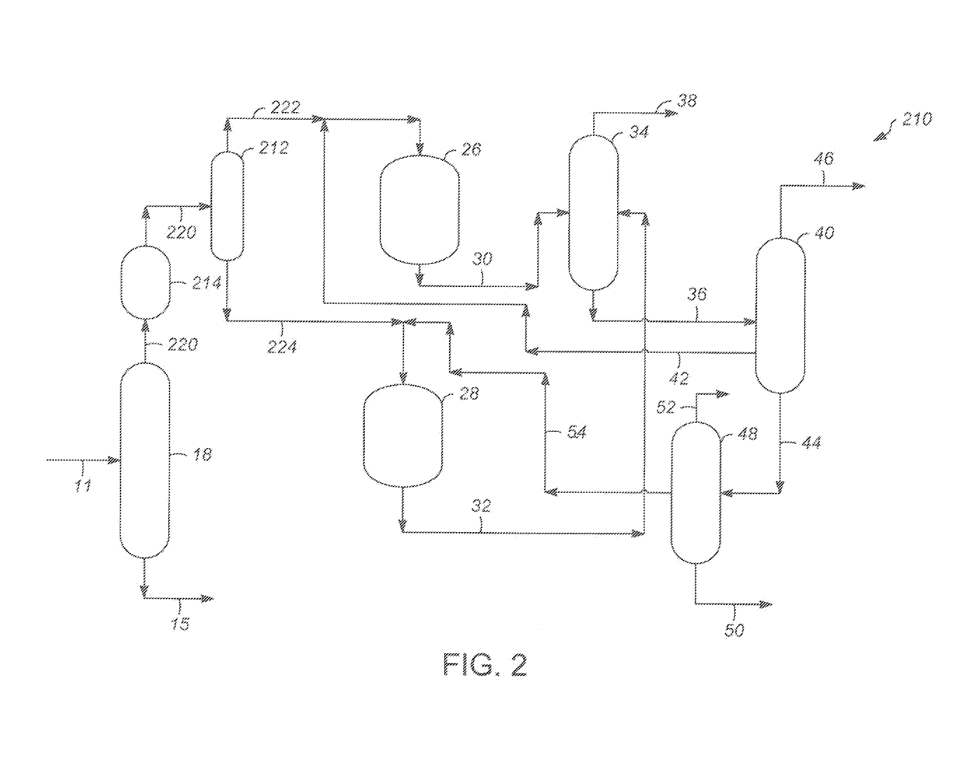

An embodiment of a process for isomerizing hydrocarbons will now be addressed with reference to an exemplary apparatus 10 for isomerizing hydrocarbons as shown in FIG. 1. In accordance with the process and as shown in FIG. 1, a first hydrocarbon feed 20 is provided. The first hydrocarbon feed 20 includes hydrocarbons that have from 5 to 7 carbon atoms, and may further include various other hydrocarbons including hydrocarbons having 8 or more carbon atoms. The hydrocarbons included in the first hydrocarbon feed 20 may be aromatic, aliphatic saturated, aliphatic unsaturated, or cyclic hydrocarbons. The first hydrocarbon feed 20 is generally depleted of hydrocarbons that have less than 5 carbon atoms since such hydrocarbons are generally employed in other industrial processes. The first hydrocarbon feed 20 may include fresh feed, recycled feed, or by-products from refining of other fractions derived from petroleum. In other embodiments, the first hydrocarbon feed 20 may be a fraction that contains hydrocarbons only having from 5 to 7 carbon atoms, such as a first hydrocarbon feed 220 discussed in more detail with reference to FIG. 2 below.

In accordance with the processes described herein, the first hydrocarbon feed 20 is fractionated to produce a first separated stream 22 that includes hydrocarbons having from 5 to 6 carbon atoms and a second separated stream 24 having hydrocarbons having 7 carbon atoms. Additionally, in an embodiment and as shown in FIG. 1, the first hydrocarbon feed 20 is further fractionated into a first heavy fractionation stream 15 that includes aromatic hydrocarbons having at least 7 carbon atoms, as well as aliphatic hydrocarbons having at least 8 carbon atoms. Fractionation may be conducted through conventional distillation in a first fractionation unit 12, which may include one or more distillation columns that are adapted to fractionate the first hydrocarbon feed 20 to produce the first separated stream 22 and the second separated stream 24, as well as optionally the first heavy fractionation stream 15.

It is to be appreciated that compounds included in the respective streams 22, 24, 15 generally boil at about the same temperature, and it is also to be appreciated that aromatic hydrocarbons generally boil at substantially the same temperature as aliphatic hydrocarbons that have one more carbon atom (e.g., benzene generally boils with linear or branched heptane; toluene generally boils with linear or branched octane). In embodiments, the first hydrocarbon feed 20 is fractionated to provide hydrocarbons having from 5 to 6 carbon atoms in the first separated stream 22 while substantially excluding hydrocarbons having more than 6 carbon atoms for reasons to be described below. By “substantially excluding”, it is meant that the hydrocarbons having 7 carbon atoms may be present in amounts of less than about 1 wt % based on the total weight of the first separated stream 22. The second separated stream 24 is generally taken as a side cut to include aliphatic hydrocarbons having 7 carbon atoms, and also generally includes hydrocarbons having 5 or 6 carbon atoms since separation of C5 to C7 hydrocarbons may be difficult with the presence of high amounts of hydrocarbons having 5 or 6 carbon atoms being acceptable in the second separated stream 24 for reasons described below. In fact, hydrocarbons that have 5 or 6 carbon atoms may be present in unrestricted amounts in the second separated stream 24 provided that hydrocarbons that have 7 carbon atoms are also present in the second separated stream 24 and are substantially excluded from the first separated stream 22. The first heavy fractionation stream 15 is generally taken to include aromatic hydrocarbons having at least 7 carbon atoms, although it is to be appreciated that some aromatic hydrocarbons having at least 7 carbons may be present in the second separated stream 24 in accordance with limitations of conventional fractionation techniques.

The first separated stream 22 is isomerized in the presence of a first isomerization catalyst and hydrogen under first isomerization conditions to produce a first isomerized stream 30, and the second separated stream 24 is isomerized in the presence of a second isomerization catalyst and hydrogen under second isomerization conditions that are different from the first isomerization conditions to produce a second isomerized stream 32. Isomerization is a common process in the refining of hydrocarbons, and is typically employed to increase octane values of linear paraffins by converting the linear paraffins to branched paraffins in the presence of hydrogen and isomerization catalysts. In embodiments and as shown in FIG. 1, a first isomerization unit 26 is in fluid communication with the first fractionation unit 12 and is adapted to receive and isomerize the first separated stream 22 in the presence of the first isomerization catalyst and hydrogen to produce the first isomerized stream 30. Additionally and as also shown in FIG. 1, a second isomerization unit 28 that is separate from the first isomerization unit 26 is in fluid communication with the first fractionation unit 12 and is adapted to receive and isomerize the second separated stream 24 in the presence of the second isomerization catalyst and hydrogen to produce the second isomerized stream 32.

The first isomerization catalyst and the second isomerization catalyst are the same type of isomerization catalyst. However, the first separated stream 22 and the second separated stream 24 are separately isomerized because hydrocarbons having 7 carbon atoms generally crack under conditions that are ideal for isomerizing hydrocarbons that have 5 or 6 carbon atoms. Isomerization of the second separated stream 24 is generally conducted at less severe conditions than the first separated stream 22, and the hydrocarbons having 5 or 6 carbon atoms are generally inert under the less severe conditions at which the second separated stream 24 is isomerized. In embodiments, the second separated stream 24 is isomerized at a higher space velocity than a space velocity of the first separated stream 22. While particular space velocities at which the first separated stream 22 and the second separated stream 24 are isomerized may vary depending upon numerous variables including particular isomerization catalysts and isomerization units used, as well as isomerization temperature, typical space velocities range from about 0.5 to about 20. The first separated stream 22 may be isomerized at lower values within the aforementioned range, such as from about 0.5 to about 6, and the second separated stream 24 may be isomerized at higher values within the aforementioned range, such as from about 2 to about 20. In addition to or as an alternative to different space velocities, the second separated stream 24 may be isomerized at a lower isomerization temperature than an isomerization temperature of the first separated stream 22. Isomerization temperatures are also subject to the above-referenced variables, although typical isomerization temperatures range from about 60° C. to about 200° C. The first separated stream 22 may be isomerized at higher values within the aforementioned range, such as from about 100° C. to about 200° C., and the second separated stream 24 may be isomerized at lower values within the aforementioned range, such as from about 60° C. to about 180° C.

As set forth above, the first isomerization catalyst and the second isomerization catalyst are the same type of isomerization catalyst. By employing the same type of isomerization catalyst, process efficiencies may be maximized by employing common unit operations where possible. In an embodiment, chlorided alumina is employed as the first isomerization catalyst and the second isomerization catalyst. The chlorided alumina may include, for example, chlorided platinum alumina catalyst. The alumina can be an anhydrous gamma-alumina, although other aluminas may be utilized. In addition to platinum, the isomerization catalysts may optionally include one or more of palladium, germanium, ruthenium, rhodium, osmium, and iridium. The isomerization catalysts may contain from about 0.1 to about 0.25 wt % platinum, and optionally from about 0.1 to about 0.25 wt % of one or more of palladium, germanium, ruthenium, rhodium, osmium, and iridium, based on the total weight of the isomerization catalysts. Because chlorided alumina catalysts generally require drying of the stream to be isomerized, in an embodiment and as shown in FIG. 1, the first separated stream 22 and the second separated stream 24 are dried, and drying may be conducted through conventional drying techniques in a first dryer 14 for the first separated stream 22 and a second dryer 16 for the second separated stream 24, with the first separated stream 22 dried independent of the second separated stream 24.

As shown in FIG. 1, after isomerization, the first isomerized stream 30 and the second isomerized stream 32 are stabilized together to produce a light stream 38 that includes hydrocarbons having less than or equal to 4 carbon atoms and a stabilized stream 36 that includes branched hydrocarbons. It is to be appreciated that the light stream 38 may also include hydrogen, although minor amounts of hydrogen are generally present when the chlorided alumina is employed as the first isomerization catalyst and the second isomerization catalyst. Further, in embodiments where chlorided alumina isomerization catalysts are employed, the light stream 38 further includes chlorides. It is to be appreciated that, in addition to branched hydrocarbons, the stabilized stream 36 may also include residual linear hydrocarbons having from 5 to 7 carbon atoms as well as naphthenes having 6 or 7 carbon atoms. Referring to FIG. 1, stabilization may be conducted in a stabilizer 34 that is in fluid communication with the first isomerization unit 26 and the second isomerization unit 28 for receiving the first isomerized stream 30 and the second isomerized stream 32. Because the first isomerized stream 30 and the second isomerized stream 32 are stabilized together, the chlorides from the respective isomerized streams 30, 32 may be removed in a single unit operation for downstream remediation, thereby consolidating removal and remediation of the chlorides from the respective isomerized streams 30, 32.

In an embodiment and as shown in FIG. 1, the stabilized stream 36 is fractionated into a first product stream 46 that includes branched hydrocarbons that have less than or equal to 6 carbon atoms and linear hydrocarbons that have less than or equal to 5 carbon atoms, a normal hexane-enriched stream 42, and a second heavy fractionation stream 44 that includes hydrocarbons having at least 7 carbon atoms. In addition to normal hexane, the normal hexane-enriched stream 42 may also include 2-methyl pentane, 3-methylpentane, and some cyclic hydrocarbons having 6 carbon atoms. The second heavy fractionation stream 44 may also include some cyclic hydrocarbons having 6 carbon atoms. Fractionation of the stabilized stream 36 may be conducted in a deisohexanizer 40 that is in fluid communication with the stabilizer 34 for receiving the stabilized stream 36, and the deisohexanizer 40 may be operated through conventional techniques to effectuate separation of the first product stream 46, the normal hexane-enriched stream 42, and the second heavy fractionation stream 44. Because the common stabilizer 34 is employed in this embodiment, the deisohexanizer 40 is employed to separate components that originate from both the first isomerized stream 30 and the second isomerized stream 32. Further, isomerization of hydrocarbons having 7 carbon atoms generally involves some cracking to hydrocarbons having 5 or 6 carbon atoms, and use of a single deisohexanizer 40 enables the cracked hydrocarbons having 5 or 6 carbon atoms to be recovered along with isomerized species from the first isomerized stream 30. Further still, the use of a single deisohexanizer 40 also remediates imperfect fractionation of the first hydrocarbon feed 20 and renders immaterial the inclusion of hydrocarbons that have 5 or 6 carbon atoms in the second separated stream 24 that is subject to less severe isomerization conditions than the first separated stream 22. Generally, significantly less hydrocarbons having 5 carbon atoms are present in the second separated stream 24 than hydrocarbons having 6 carbon atoms. Further, the linear hydrocarbons that have 6 carbon atoms as well as methyl pentanes are separated in the normal hexane-enriched stream 42 and are available for further isomerization. In an embodiment and as shown in FIG. 1, the normal hexane-enriched stream 42 is combined with the first separated stream 22 and the normal hexane-enriched stream 42 is isomerized together with the first separated stream 22.

In an embodiment and as shown in FIG. 1, the second heavy fractionation stream 44 is fractionated into a second product stream 52 that includes branched hydrocarbons that have less than or equal to 7 carbon atoms, a normal heptane-enriched stream 54, and a third heavy fractionation stream 50 that includes cyclic hydrocarbons having at least 7 carbon atoms. In addition to normal heptane, the normal heptane-enriched stream 54 may also include various methyl hexanes. Fractionation of the second heavy fractionation stream 44 may be conducted in a deisoheptanizer 48 that is in fluid communication with the deisohexanizer 40 for receiving the second heavy fractionation stream 44, and the deisoheptanizer 48 may be operated through conventional techniques to effectuate separation of second heavy fractionation stream into the second product stream 52, the normal heptane-enriched stream 54, and the third heavy fractionation stream 50. The normal heptane-enriched stream 54 is available for further isomerization. In an embodiment and as shown in FIG. 1, the normal heptane-enriched stream 54 is combined with the second separated stream 24 and the normal heptane-enriched stream 54 is isomerized together with the second separated stream 24.

Another embodiment of a process for isomerizing hydrocarbons will now be addressed with reference to another exemplary apparatus 210 for isomerizing hydrocarbons as shown in FIG. 2. In accordance with the process and as shown in FIG. 2, chlorided alumina is again employed as the first isomerization catalyst and the second isomerization catalyst, with drying incorporated into the process. However, in this embodiment, drying is consolidated into a single drying unit 214 by drying a first hydrocarbon feed 220 prior to fractionating the first hydrocarbon feed 220 to produce a first separated stream 222 that includes hydrocarbons having from 5 to 6 carbon atoms and a second separated stream 224 that includes hydrocarbons having 7 carbon atoms. Referring to FIG. 2, a mixed hydrocarbon stream 11 that includes hydrocarbons having from 5 carbon atoms to at least 8 carbon atoms is fractionated in an upstream fractionation unit 18 to produce the first hydrocarbon feed 220 and a first heavy fractionation stream 15 that includes aromatic hydrocarbons having at least 7 carbon atoms. The mixed hydrocarbon stream 11 is effectively fractionated to first separate the first heavy fractionation stream 15 prior to separating the species that ultimately are included in the first separated stream 222 and the second separated stream 224, with those species included together in the first hydrocarbon feed 220 for consolidated drying in the single drying unit 214. In this embodiment, the first hydrocarbon feed 220 includes hydrocarbons that have from 5 to 7 carbon atoms, and is substantially free of aromatic hydrocarbons having 7 or more carbon atoms and aliphatic hydrocarbons having 8 or more carbon atoms. After drying, the first hydrocarbon feed 220 is fractionated to produce the first separated stream 222 and the second separated stream 224. Fractionation of the first hydrocarbon feed 220 may be conducted in a first fractionation unit 212 in the same manner as described above for the embodiment of FIG. 1.

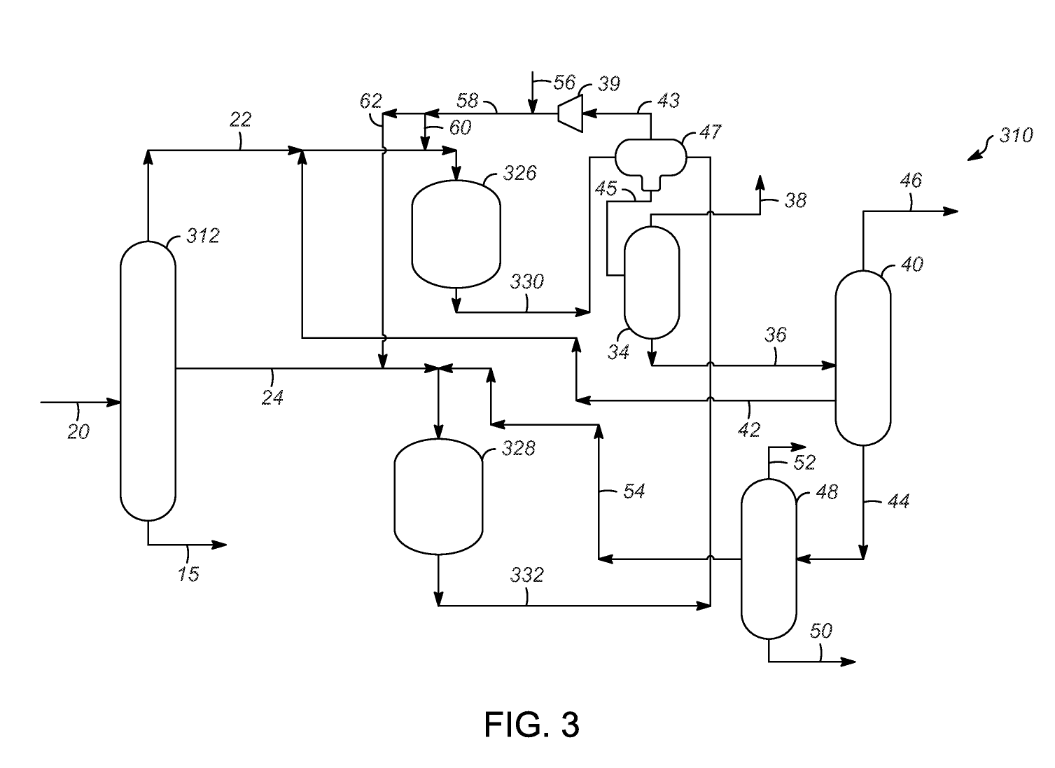

Another embodiment of a process for isomerizing hydrocarbons will now be addressed with reference to another exemplary apparatus 310 for isomerizing hydrocarbons as shown in FIG. 3. In this embodiment, the first hydrocarbon feed 20 is fractionated in the same manner as described above in the embodiment of FIG. 1 to produce the first separated stream 22 and the second separated stream 24. However, in this embodiment, a zirconia-containing catalyst is employed as the first isomerization catalyst in a first isomerization unit 326 and the second isomerization catalyst in a second isomerization unit 328. Suitable zirconia-containing catalysts include, for example, noble metal such as platinum on sulfated or tungstated zirconia. Unlike chlorided alumina, zirconia-containing catalysts are not as selective as chlorided alumina and generally incorporate a hydrogen recycle, and the apparatus 310 of FIG. 3 incorporates additional supporting unit operations to facilitate hydrogen recycle to both the first isomerization unit 326 and the second isomerization unit 328 in a consolidated manner.

In this embodiment, after isomerizing the first separated stream 22 and the second separated stream 24, a first isomerized stream 330 and a second isomerized stream 332 are produce, but with the first isomerized stream 330 and the second isomerized stream 332 including significantly more hydrogen than corresponding streams produced in the presence of chlorided alumina isomerization catalysts. Liquid/vapor separation of the first isomerized stream 330 and the second isomerized stream 332 is conducted together, i.e., in the same unit operation, to produce a vapor stream 43 that includes hydrogen and a liquid stream 45 that includes hydrocarbons. Liquid/vapor separation may be conducted in a liquid/vapor separation unit 47 through conventional separation techniques. The liquid stream 45 may be stabilized in the stabilizer 34 in the same manner as set forth above, except that the stabilizer 34 is in fluid communication with the liquid/vapor separation unit 47 and is adapted to stabilize the first isomerized stream 330 and the second isomerized stream 332 together. In this embodiment, the vapor stream 43 is further processed to recover and recycle hydrogen to the isomerization units 326, 328. The vapor stream 43 may be compressed in a compressor 39 to produce a compressed hydrogen stream 58, and makeup hydrogen 56 may be mixed with the compressed hydrogen stream 58 as needed. As shown in FIG. 3, the compressed hydrogen stream 58 may be split into a first recycle hydrogen stream 60 and a second recycle hydrogen stream 62. The first recycle hydrogen stream 60 may be combined with the first separated stream 22 and the second recycle hydrogen stream 62 may be combined with the second separated stream 24. In this regard, recycled hydrogen may be provided to both the first separated stream 22 and the second separated stream 24 with consolidated supporting unit operations by employing the common liquid/vapor separation unit 47, the single compressor 39, and by splitting the resulting compressed hydrogen stream 58, thereby maximizing process efficiency and minimizing costs.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A process for isomerizing hydrocarbons, wherein the process comprises:
providing a first hydrocarbon feed comprising hydrocarbons having from 5 to 7 carbon atoms;
fractionating the first hydrocarbon feed to produce a first separated stream comprising hydrocarbons having from 5 to 6 carbon atoms and a second separated stream comprising hydrocarbons having 7 carbon atoms;
isomerizing the first separated stream in the presence of a first isomerization catalyst and hydrogen under first isomerization conditions to produce a first isomerized stream;
isomerizing the second separated stream in the presence of a second isomerization catalyst and hydrogen under second isomerization conditions different from the first isomerization conditions to produce a second isomerized stream, wherein the first isomerization catalyst is the same type of isomerization catalyst as the second isomerization catalyst; and
stabilizing the first isomerized stream and the second isomerized stream together to produce a light stream comprising hydrocarbons having less than or equal to 4 carbon atoms and a stabilized stream comprising branched hydrocarbons.

2. The process of claim 1, wherein isomerizing the second separated stream comprises isomerizing the second separated stream at a higher space velocity than a space velocity of the first separated stream.

3. The process of claim 2, wherein isomerizing the second separated stream comprises isomerizing the second separated stream at a lower isomerization temperature than an isomerization temperature of the first separated stream.

4. The process of claim 1, further comprising fractionating the stabilized stream into a first product stream comprising branched hydrocarbons having less than or equal to 6 carbon atoms and linear hydrocarbons having less than or equal to 5 carbon atoms, a normal hexane-enriched stream, and a second heavy fractionation stream comprising hydrocarbons having at least 7 carbon atoms.

5. The process of claim 4, further comprising isomerizing the normal hexane-enriched stream together with the first separated stream.

6. The process of claim 4, further comprising fractionating the second heavy fractionation stream into a second product stream comprising branched hydrocarbons having less than or equal to 7 carbon atoms, a normal heptane-enriched stream, and a third heavy fractionation stream comprising cyclic hydrocarbons having at least 7 carbon atoms.

7. The process of claim 6, further comprising isomerizing the normal heptane-enriched stream together with the second separated stream.

8. The process of claim 1, wherein isomerizing the first separated stream and isomerizing the second separated stream comprises isomerizing the first separated stream in the presence of chlorided alumina as the first isomerization catalyst and isomerizing the second separated stream in the presence of chlorided alumina as the second isomerization catalyst.

9. The process of claim 8, wherein providing the first hydrocarbon feed comprises fractionating a mixed hydrocarbon stream comprising hydrocarbons having from 5 carbon atoms to at least 8 carbon atoms to produce the first hydrocarbon feed and a first heavy fractionation stream comprising aromatic hydrocarbons having at least 7 carbon atoms.

10. The process of claim 9, further comprising drying the first hydrocarbon feed prior to fractionating the first hydrocarbon feed.

11. The process of claim 8, further comprising drying the first separated stream and drying the second separated stream independent of drying the first separated stream.

12. The process of claim 1, wherein isomerizing the first separated stream and isomerizing the second separated stream comprises isomerizing the first separated stream in the presence of a zirconia-containing catalyst as the first isomerization catalyst and isomerizing the second separated stream in the presence of a zirconia-containing catalyst as the second isomerization catalyst.

13. The process of claim 12, further comprising conducting liquid/vapor separation of the first isomerized stream and the second isomerized stream together to produce a vapor stream comprising hydrocarbons having less than or equal to 4 carbon atoms and hydrogen and a liquid stream comprising branched hydrocarbons.

14. The process of claim 13, further comprising compressing the vapor stream to produce a compressed hydrogen stream.

15. The process of claim 14, further comprising splitting the compressed hydrogen stream into a first recycle hydrogen stream and a second recycle hydrogen stream.

16. The process of claim 15, further comprising combining the first recycle hydrogen stream and the first separated stream and combining the second recycle hydrogen stream and the second separated stream.

17. A process for isomerizing hydrocarbons, wherein the process comprises:
fractionating a first hydrocarbon feed to produce a first separated stream comprising hydrocarbons having from 5 to 6 carbon atoms and a second separated stream comprising hydrocarbons having 7 carbon atoms;
isomerizing the first separated stream in the presence of a first isomerization catalyst and hydrogen under first isomerization conditions to produce a first isomerized stream;
isomerizing the second separated stream in the presence of a second isomerization catalyst and hydrogen under second isomerization conditions different from the first isomerization conditions to produce a second isomerized stream, wherein the first isomerization catalyst is the same type of isomerization catalyst as the second isomerization catalyst;
stabilizing the first isomerized stream and the second isomerized stream together to produce a light stream comprising hydrocarbons having less than or equal to 4 carbon atoms and a stabilized stream comprising branched hydrocarbons;
fractionating the stabilized stream into a first product stream comprising branched hydrocarbons having less than or equal to 6 carbon atoms and linear hydrocarbons having less than or equal to 5 carbon atoms, a normal hexane-enriched stream, and a second heavy fractionation stream comprising hydrocarbons having at least 7 carbon atoms, wherein the normal hexane-enriched stream is combined with the first separated stream; and
fractionating the second heavy fractionation stream into a second product stream comprising branched hydrocarbons having less than or equal to 7 carbon atoms, a normal heptane-enriched stream, and a third heavy fractionation stream comprising cyclic hydrocarbons having at least 7 carbon atoms, wherein the normal heptane-enriched stream is combined with the second separated stream.

18. An apparatus for isomerizing hydrocarbons, wherein the apparatus comprises:
a first fractionation unit adapted to fractionate a first hydrocarbon feed comprising hydrocarbons having from 5 to 7 carbon atoms to produce a first separated stream comprising hydrocarbons having from 5 to 6 carbon atoms and a second separated stream comprising hydrocarbons having 7 carbon atoms;
a first isomerization unit in fluid communication with the first fractionation unit and adapted to receive and isomerize the first separated stream in the presence of a first isomerization catalyst and hydrogen under first isomerization conditions to produce a first isomerized stream;
a second isomerization unit in fluid communication with the first fractionation unit and adapted to receive and isomerize the second separated stream in the presence of a second isomerization catalyst and hydrogen under second isomerization conditions different from the first isomerization conditions to produce a second isomerized stream, wherein the first isomerization catalyst is the same type of isomerization catalyst as the second isomerization catalyst; and
a stabilizer adapted to stabilize the first isomerized stream and the second isomerized stream together to produce a light stream comprising hydrocarbons having less than or equal to 4 carbon atoms and a stabilized stream comprising branched hydrocarbons.

19. The apparatus of claim 18, further comprising a deisohexanizer in fluid communication with the stabilizer for receiving the stabilized stream, wherein the deisohexanizer is adapted to fractionate the stabilized stream into a first product stream comprising branched hydrocarbons having less than or equal to 6 carbon atoms and linear hydrocarbons having less than or equal to 5 carbon atoms, a normal hexane-enriched stream, and a second heavy fractionation stream comprising hydrocarbons having at least 7 carbon atoms.

20. The apparatus of claim 19, further comprising a deisoheptanizer in fluid communication with the deisohexanizer for receiving the second heavy fractionation stream, wherein the deisoheptanizer is adapted to fractionate the second heavy fractionation stream into a second product stream comprising branched hydrocarbons having less than or equal to 7 carbon atoms, a normal heptane-enriched stream, and a third heavy fractionation stream comprising cyclic hydrocarbons having at least 7 carbon atoms, wherein the normal heptane-enriched stream is combined with the second separated stream.
